(19) United States
(12) Patent Application Publication
(10) Pub. No.: US 2004/0128079 A1
(43) Pub. Date: Jul. 1, 2004

(54) Artificial intelligent system for protein superfamily classification

(76) Inventors: Jia-Jye Shyu, Hsinchu (TW); Kuan-Jui Ho, Hsinchu (TW); Chung-Jen Ou, Hsinchu (TW)

Correspondence Address: BIRCH STEWART KOLASCH & BIRCH, PO BOX 747, FALLS CHURCH, VA, 22040-0747, US

(21) Appl. No.: 10/612,965

(22) Filed: Jul. 7, 2003

(30) Foreign Application Priority Data
Dec. 31, 2002 (TW) 91138071

(51) Int. Cl. G01N 33/50; G01N 33/48; G06F 19/00; G06G 7/00; G06E 1/00; G06F 15/18; G06E 3/00

(52) U.S. Cl. 702/19; 706/2; 706/20

(57) ABSTRACT

The AI System for protein superfamily classification is related to an artificial intelligence system for protein family classification using the fuzzy inference theory in a neural network to improve robustness, convergence and correctness. In addition, the system uses a content addressable memory to process the early phase of the classification to improve the execution speed.

### FIELD OF THE INVENTION

The invention is related to an artificial intelligent (abbreviated as AI) system for protein superfamily classification, especially to an AI system combined with the fuzzy logic system.

### BACKGROUND OF THE INVENTION

In bioinformatics technology, a classification, such as a protein superfamily classification, is an important task and costs more time and expenses. In recent years, neural network (abbreviated as NN) technology is widely used in analysis of bioinformatics.

Several research works have shown that NN technology can be used in biology chemistry family classification. For example, U.S. Pat. No. 5,845,049 has proposed a molecule sequencing method using NN technology.

Since the main coding method is N-GRAM, the amount of data and computation is quite large, hence high-end computers usually perform the classification process. Moreover, the accuracy of NN-based algorithms is not enough, and the efficiency of performing classification on computers is also not good. As a result, both drawbacks limit the applicability of the NN-based approaches.

### SUMMARY OF THE INVENTION

The invention proposes an AI system for protein family classification, uses the fuzzy logic theory in an NN system, and improves robustness, convergence and correctness by utilizing the memory and learning characteristics of NN systems, the determination expertise of the fuzzy theory which introduced the so called expert knowledge, and a content addressable memory (abbreviated as CAM) concept used to speedup input vector encoding, so that the hardware of the algorithm can work faster.

### BRIEF DESCRIPTION OF THE DRAWINGS

FIG. 1 shows the architecture of the invention.

FIG. 2A shows the search process of a traditional search approach.

FIG. 2B shows the search process of CAM.

FIG. 3A shows the first example of the combinations of a fuzzy logic system and a NN system.

FIG. 3B shows the second example of the combinations of a fuzzy logic system and a NN system.

FIG. 3C shows the third example of the combinations of a fuzzy logic system and a NN system.

### DESCRIPTION OF THE PREFERRED EMBODIMENT

The invention proposes an AI system for protein superfamily classification, which is an expert system utilizing NN technology and the fuzzy logic system. The expert system can organize the experts' knowledge and simulate the inference behavior of experts, to classify a protein family.

First, the experts' knowledge consists of linguistic variables and a fuzzy set, and a fuzzy expert system is built by the derived linguistic variables and fuzzy set. The inference process of the fuzzy logic can be represented by a resolution function. Then, various algorithms in NN are used to adapt the parameters of the fuzzy expert system. The fuzzy expert system automatically updates its knowledge base, hence the fuzzy inference engine works correctly as time goes by.

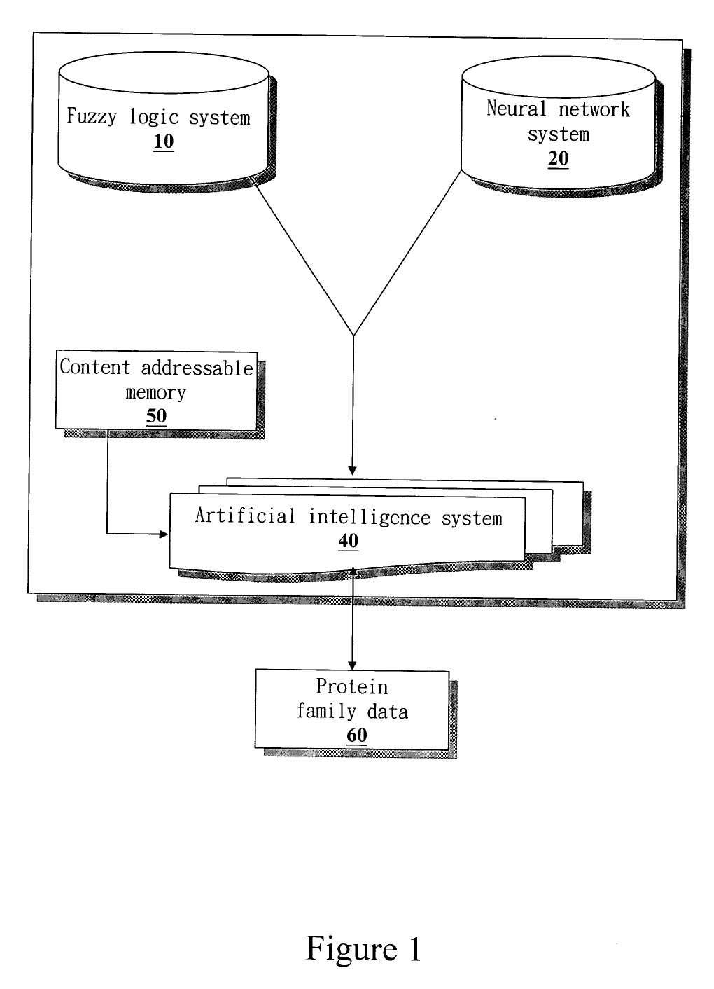

The proposed system is used to improve the efficiency of the protein family (e.g., protein super family) classification. FIG. 1 shows the architecture of the proposed system. The AI system 40 integrates a fuzzy logic system 10 into an NN system 20 to classify the protein super family sequence 60.

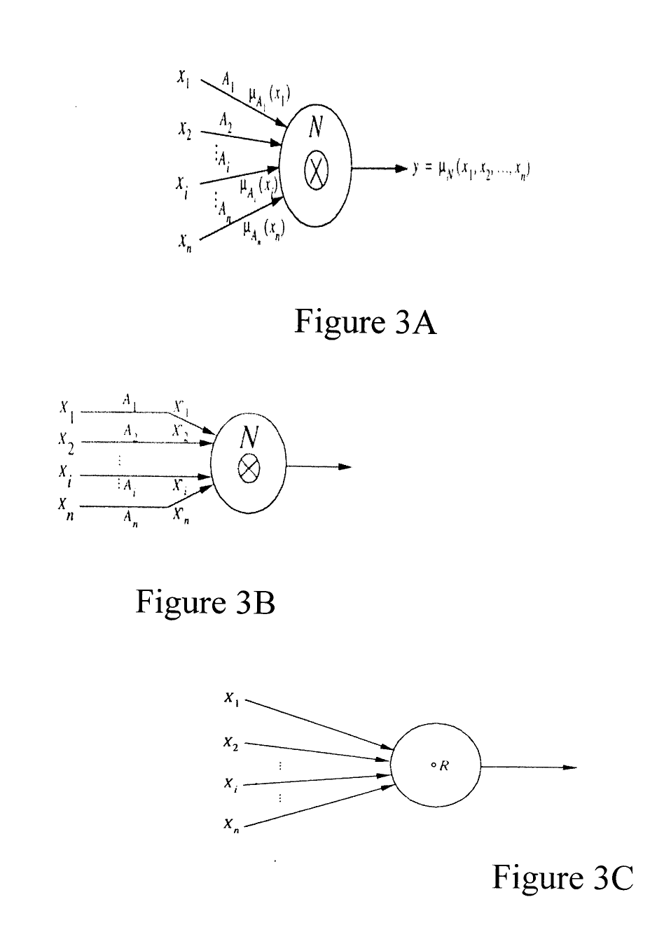

There are various combinations of a fuzzy logic system 10 and an NN system 20. FIG. 3A shows the first example of the combinations. The input data X₁˜Xₙ are processed by a fuzzy set Aᵢ. Then the results are classified by membership functions μ_{A1}˜μ_{An} and the aggregation operator \mathring{x} to obtain the classification result Y. FIG. 3B shows the second example of the combinations. It directly codes the fuzzy logic system into the NN system. The input data X₁˜Xₙ are processed by a fuzzy set Aᵢ to obtain Y=X₁\mathring{x}X₂\mathring{x} . . . . FIG. 3C shows the third example of the combinations. Multiple input Xᵢs are processed by a fuzzy transfer relation R (e.g., t-norm) to obtain the result Y.

In addition, CAM 50 concept is used in the hardware architecture to make the search process faster. It also reduces the size of the hardware architecture so that the hardware can be designed as a commercialized interface card.

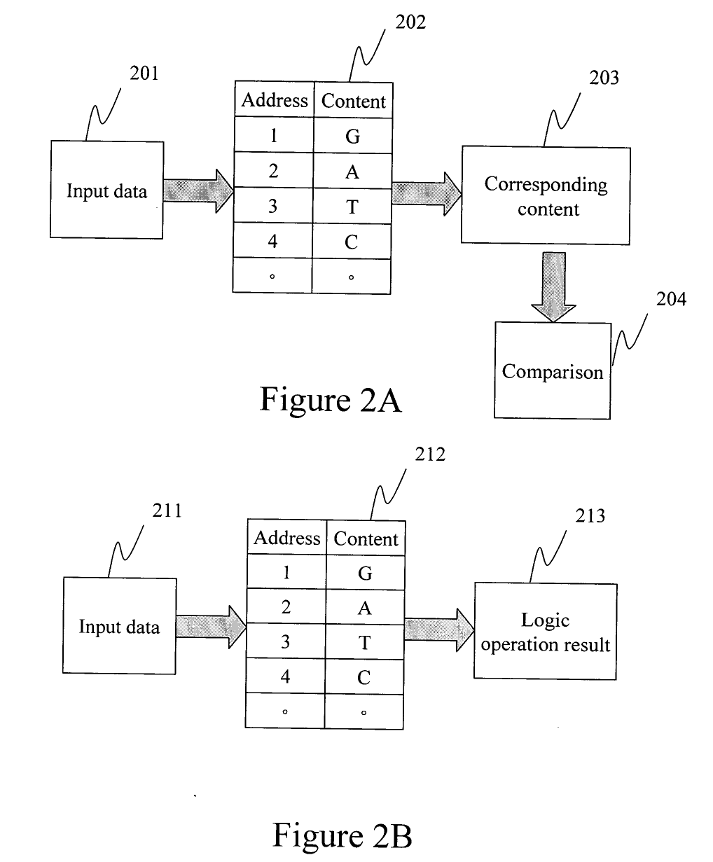

FIGS. 2A and 2B show the search processes of a traditional approach and CAM, respectively. In a traditional computer-based search method, the address to be searched is inputted (Step 201), and personal computers or other computation devices then search the address-content table 202 to obtain the corresponding content (Step 203) and compare the content (Step 204). The efficiency of the traditional approach is low, since it searches the address-content table sequentially.

In CAM, after the content is inputted, the result can be obtained by applying logical operations (Step 213) to the address-content table 212, hence the search-efficiency is improved.

The proposed AI system integrates the fuzzy inference theory into an NN system, and improves robustness, convergence and correctness by utilizing the memory and learning characteristics of NN systems, the determination expertise of the fuzzy inference theory, and a content addressable memory to make the system can be commercialized easily.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments, not departing from the spirit and scope of the invention.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. An AI system for protein superfamily sequence classification which utilizes an NN system to classify a series of protein families, characterized in:
further comprising a fuzzy logic system integrated with a NN system to improve the robustness, convergence and correctness of the system.

2. The system in accordance with claim 1, wherein the system comprises a CAM.

3. The system in accordance with claim 2, wherein the said CAM is used to compare the protein family data.

4. The system in accordance with claim 1, wherein the said fuzzy logic system can be directed coded into the said NN system.

5. The system in accordance with claim 1, wherein the input data of the NN system are weighted by a fuzzy logic before inputted into the NN system.

6. The system in accordance with claim 1, wherein the input data of the NN system is transformed into the data of the fuzzy logic.

7. An AI system for protein family classification which utilizes an NN system to classify a series of protein families, characterized in:
further comprising a fuzzy logic system to improve the robustness, convergence and correctness of the system by utilizing the CAM to compare the protein family data and integrating the fuzzy logic system and an NN system.

8. The system in accordance with claim 7, wherein the said fuzzy logic system can be directed coded into the said NN system.

9. The system in accordance with claim 7, wherein the input data of the NN system are weighted by a fuzzy logic before inputted into the NN system.

10. The system in accordance with claim 7, wherein the input data of the NN system is transformed into the data of the fuzzy logic.

11. The system in accordance with claim 7, wherein the AI system can be integrated into a portable interface card.
